(19) United States
(12) Patent Application Publication
(10) Pub. No.: US 2026/0158056 A1
(43) Pub. Date: Jun. 11, 2026

(54) USE OF LOW MOLECULAR WEIGHT HYALURONIC ACID FOR THE TREATMENT OF LUNG MUCOSAL INFLAMMATION

(71) Applicants: INSTITUT NATIONAL DE LA SANTÉ ET DE LA RECHERCHE MÉDICALE, Paris (FR); UNIVERSITÉ DE REIMS CHAMPAGNE-ARDENNE, Reims (FR); AGRO INDUSTRIE RECHERCHES ET DÉVELOPPEMENTS, Pomacle (FR); CENTRE HOSPITALIER UNIVERSITAIRE DE REIMS, Reims (FR)

(72) Inventors: Christelle CORAUX, Reims (FR); Emilie LUCZKA, Reims (FR); Myriam POLETTE, Reims (FR); Damien ADAM, Montréal, Québec (CA)

(21) Appl. No.: 18/706,622

(22) PCT Filed: Nov. 4, 2022
(86) PCT No.: PCT/EP2022/080807

§ 371 Date: May 1, 2024

(30) Foreign Application Priority Data
Nov. 5, 2021 (EP) 21306554.3

(51) Int. Cl. A61K 31/728; A61K 31/573; A61K 31/58; A61P 29/00

(52) U.S. Cl. CPC A61K 31/728; A61K 31/573; A61K 31/58; A61P 29/00

(57) ABSTRACT

In chronic inflammatory respiratory diseases such as cystic fibrosis or chronic obstructive pulmonary disease (COPD), cellular and functional balances may be disrupted, resulting in the formation of epithelial reshaping or remodelling areas with the presence of squamous metaplasia and/or basal or secretory cell hyperplasia. Thus there is a need for identifying drugs capable of treating lung mucosal inflammation. The inventors surprisingly show that hyaluronic acid (HA) with a low molecular weight, from 15,000 to 50,000 Daltons has anti-inflammatory properties on lung mucosal inflammation. The present invention thus relates to a method of treating lung mucosal inflammation in a subject suffering from cystic fibrosis or chronic obstructive pulmonary disease (COPD) comprising administering to the subject a therapeutically effective amount of hyaluronic acid having a low molecular weight, from 15,000 to 50,000 Daltons.

### FIELD OF THE INVENTION

The present invention is in the field of medicine, in particular in pulmonology.

### BACKGROUND OF THE INVENTION

The respiratory epithelium is in permanent contact with the external environment, with a total exchange surface area of approximately 100 to 130 m². In non-pathological cases, it is continuously exposed through inhalation to various pathogens or particles that can induce epithelial lesions. Facing these lesions, the airway epithelium must be able to restore its integrity through repair and regeneration mechanisms in order to regain all its functions, in particular its defence and barrier functions. Accordingly in chronic inflammatory respiratory diseases such as cystic fibrosis, chronic obstructive pulmonary disease, asthma or allergies, cellular and functional balances may be disrupted, resulting in the formation of epithelial reshaping or remodelling areas with the presence of squamous metaplasia and/or basal or secretory cell hyperplasia. Thus there is a need for identifying drugs capable of treating lung mucosal inflammation.

Hyaluronic acid (HA) is an anionic, nonsulfated glycosaminoglycan distributed widely throughout connective, epithelial, and neural tissues. Various sources of hyaluronic acid exist and typically include bacterial sources (e.g. from Streptococcus zooepidemicus) but also include avian sources (e.g. from rooster comb) and bovine sources (e.g. from bovine vitreous humor). HA participates in inflammatory reactions but its role is described as being dependent on its molecular weight. Indeed, high-molecular weight (HMW) HA has an anti-inflammatory and immunosuppressive role, while smaller fragments, called HA oligosaccharides (o-HA), are pro-inflammatory and immunostimulant (HUANG, T., CHAN, Y., CHENG, P., YOUNG, Y., LOU, P. et YOUNG, T. Increased mucociliary differentiation of human respiratory epithelial cells on hyaluronan-derivative membranes. Acta biomaterialia. 2010a. Vol. 6, n°3, p. 1191-1199; OCHOA, C., GARG, H., HALES, C. et QUINN, D. Low molecular weight hyaluronan, via AP-1 and NF-κB signalling, induces IL-8 in transformed bronchial epithelial cells. Swiss medical weekly. 2011. Vol. 141, p.13255; RUPPERT, S., HAWN, T., ARRIGONI, A., WIGHT, T. et BOLLYKY, P. Tissue integrity signals communicated by high-molecular weight hyaluronan and the resolution of inflammation. Immunologic research. 2014. Vol. 58, n°2-3, p. 186-192). It has been shown on chondrocytes that an HA and resveratrol gel inhibits LPS-induced inflammation of pathogens and reduces IL-1B secretion (SHEU, S., CHEN, W., SUN, J., LIN, F. et WU, T. Biological characterization of oxidized hyaluronic acid resveratrol hydrogel for cartilage tissue engineering. Journal of biomedical materials research. Part A. 2013. Vol. 101, n°12, p.3457-3466). A recent study, in homozygous F508del-CFTR mice expressing the β subunit of the ENaC channel, demonstrated that inhalation of a high-molecular weight HA solution exerted an anti-inflammatory effect, objectified by the decrease in protein expression of pro-inflammatory cytokines such as TNF-α and MIP-2 (macrophage inflammatory protein-2) (GAVINA, M., LUCIANI, A., VILLELLA, V., ESPOSITO, S., FERRARI, E., BRESSANI, I., CASALE, A., BRUSCIA, E., MAIURI, L. et RAIA, V. Nebulized hyaluronan ameliorates lung inflammation in cystic fibrosis mice. Pediatric pulmonology. 2013. Vol. 48, n°8, p.761-771).

WO2004050187 as well as WO2009024677 disclose pharmaceutical uses of HA with a low molecular weight, from 30,000 to 45,000 Daltons for the treatment of respiratory diseases of the upper airways. In particular, WO2004050187 teaches that said HA could be suitable for repairing the epithelium and to change the respiratory mucus surface properties to promote its transport by the ciliary activity. WO2009024677 discloses the use of said HA for restoring the defense functions of the junction complexes after an attack on the epithelium.

### SUMMARY OF THE INVENTION

The present invention is defined by the claims. In particular, the present invention relates to methods of treating lung mucosal inflammation.

### DETAILED DESCRIPTION OF THE INVENTION

The inventors surprisingly show that HA with a low molecular weight, from 15,000 to 50,000 Daltons has anti-inflammatory properties on lung mucosal inflammation in a subject suffering from an inflammatory disease (e.g. cystic fibrosis or COPD).

Accordingly, the first object of the present invention relates to a method of treating lung mucosal inflammation in a subject suffering from cystic fibrosis or chronic obstructive pulmonary disease (COPD) comprising administering to the subject a therapeutically effective amount of hyaluronic acid having a low molecular weight, from 15,000 to 50,000 Daltons.

As used herein, the term “subject” denotes a mammal, such as a rodent, a feline, a canine, and a primate. Particularly, the subject according to the invention is a human. The term “subject” encompasses the term “patient”.

As used herein, the term “inflammation” has its general meaning in the art and is used to describe the fundamental pathological process consisting of a dynamic complex of cytologic and histologic reactions that occur in tissues in response to an injury or abnormal stimulation caused by a physical, chemical or biologic agent (e.g. bacterium, virus . . . ) including the local reactions and resulting morphologic changes, the destruction or removal of the injurious material, and the responses that lead to repair and healing. The so-called cardinal signs of inflammation are swelling, pain and, in certain cases, inhibited or lost function of the target organ. The swelling ordinary occurs from the congestion and exudation; pressure on (or stretching of) nerve endings as well as changes in osmotic pressure and pH which may lead to significant pain; the disturbance in function may result in impairment in movement or the actual destruction of an anatomic part or organ. In some embodiments, the inflammation is localized in pulmonary tract, especially the lungs, and is favorably treated by the method of the present invention. Especially inflammation localized to the oral mucosa e.g. buccal and sublingual; nasal mucosa; lung mucosa; bronchial mucosa is favorably treated by the method of the present invention.

As used herein, the expression “lung mucosal inflammation” has its general meaning in the art and refers to swelling or irritation of the lung mucosa. As used, the term “mucosa” has its general meaning in the art and denotes the moist tissue lining body cavities which secretes mucous and covered with epithelium.

In some embodiments, the subject suffers from a mucosal inflammatory disease that affects the respiratory system and typically includes cystic fibrosis and chronic obstructive pulmonary disease.

Thus, in some embodiments, the subject suffers from cystic fibrosis.

As used herein the term “cystic fibrosis” has its general meaning in the art and refers to an inherited autosomal disease associated with mutations to the gene encoding the cystic fibrosis transmembrane conductor regulator (CFTR). The method of the invention may be performed for any type of cystic fibrosis such as revised in the World Health Organisation Classification of cystic fibrosis and selected from the E84 group: mucoviscidosis, Cystic fibrosis with pulmonary manifestations, Cystic fibrosis with intestinal manifestations and Cystic fibrosis with other manifestations. In some embodiments, the subject harbours at least one mutation in the CFTR gene, including, but not limited to F508del-CFTR, R117H CFTR, and G551D CFTR (see, e.g., http://www.genet.sickkids.on.ca/cftr, for CFTR mutations).

In some embodiments, the subject suffers from chronic obstructive pulmonary disease (COPD).

As used herein, the term “COPD” refers to chronic obstructive pulmonary disease. The term “COPD” is generally applied to chronic respiratory disease processes characterized by the persistent obstruction of bronchial airflow. COPD patients can suffer from conditions such as bronchitis or emphysema.

In some embodiments, the lung mucosal inflammation can result from a lung infection.

As used herein, the term “lung infection” has its general meaning in the art and means the invasion of lung tissues of a patient by disease-causing microorganisms, their multiplication and the reaction of lung tissues to these microorganisms and the toxins that they produce. In some embodiments, the patient suffers from a chronic lung infection.

As used herein, the term “chronic infection” refers to a long-term infection which may be an apparent, unapparent or latent infection. In some embodiments, the patient suffers from an acute lung infection.

In some embodiments, the lung infection is a bacterial infection, such as bacterial pneumonia.

In some embodiments, the bacterial infection is caused by a bacterium selected from the group consisting of Streptococcus pneumoniae (also referred to as pneumococcus), Staphylococcus aureus, Streptococcus agalactiae, Streptococcus pyogenes, Haemophilus influenzae, Haemophilus parainfluenzae, Klebsiella pneumoniae, Escherichia coli, Pseudomonas aeruginosa, Moraxella catarrhalis, Chlamydophila pneumoniae, Mycoplasma pneumoniae, Legionella pneumophila, Serratia marcescens, Burkholderia cepacia, Burkholderia pseudomallei, Bacillus anthracis, Bacillus cereus, Bordatella pertussis, Stenotrophomonas maltophilia, a bacterium from the citrobacter family, a bacterium from the ecinetobacter family, and Mycobacterium tuberculosis or Mycobacterium abscessus. In some embodiments, the lung infection is a fungal infection. In some embodiments, the fungal infection is caused by a fungus selected from the group consisting of Histoplasma capsulatum, Cryptococcus neoformans, Pneumocystis jiroveci, Coccidioides immitis, Candida albicans, and Pneumocystis jirovecii (which causes pneumocystis pneumonia (PCP), also called pneumocystosis) or Aspergillus fumigatus. In some embodiments, the lung infection is a viral infection, such viral pneumonia. In some embodiments, the viral infection is caused by a virus selected from the group consisting of influenza virus (e.g., Influenza virus A, Influenza virus B), respiratory syncytial virus, adenovirus, metapneumovirus, cytomegalovirus, parainfluenza virus (e.g., hPIV-1, hPIV-2, hPIV-3, hPIV-4), rhinovirus, coxsackie virus, echo virus, herpes simplex virus, coronavirus (SARS-coronavirus such as SARS-Cov1 or SARS-Cov2), and smallpox. In some embodiments, the viral lung infection may be due to a member of the Pneumoviridae, Paramyxoviridae and/or Coronaviridae families are in particular selected from the group consisting of upper and lower respiratory tract infections due to: human respiratory syncytial virus (hRSV), type A and type B, human metapneumovirus (hMPV) type A and type B; parainfluenza virus type 3 (PIV-3), measles virus, endemic human coronaviruses (HCoV-229E, -NL63, -OC43, and -HKU1), severe acute respiratory syndrome (SARS) and Middle-East respiratory syndrome (MERS) coronaviruses. In particular, the method of the present invention is suitable for the treatment of Severe Acute Respiratory Syndrome (SARS). More particularly, the method of the present invention is suitable for the treatment of lung mucosal inflammation in patients suffering from COVID-19.

As used herein, the term “treatment” or “treat” refer to both prophylactic or preventive treatment as well as curative or disease modifying treatment, including treatment of patient at risk of contracting the disease or suspected to have contracted the disease as well as patients who are ill or have been diagnosed as suffering from a disease or medical condition, and includes suppression of clinical relapse. The treatment may be administered to a subject having a medical disorder or who ultimately may acquire the disorder, in order to prevent, cure, delay the onset of, reduce the severity of, or ameliorate one or more symptoms of a disorder or recurring disorder, or in order to prolong the survival of a subject beyond that expected in the absence of such treatment. By “therapeutic regimen” is meant the pattern of treatment of an illness, e.g., the pattern of dosing used during therapy. A therapeutic regimen may include an induction regimen and a maintenance regimen. The phrase “induction regimen” or “induction period” refers to a therapeutic regimen (or the portion of a therapeutic regimen) that is used for the initial treatment of a disease. The general goal of an induction regimen is to provide a high level of drug to a patient during the initial period of a treatment regimen. An induction regimen may employ (in part or in whole) a “loading regimen”, which may include administering a greater dose of the drug than a physician would employ during a maintenance regimen, administering a drug more frequently than a physician would administer the drug during a maintenance regimen, or both. The phrase “maintenance regimen” or “maintenance period” refers to a therapeutic regimen (or the portion of a therapeutic regimen) that is used for the maintenance of a patient during treatment of an illness, e.g., to keep the patient in remission for long periods of time (months or years). A maintenance regimen may employ continuous therapy (e.g., administering a drug at a regular intervals, e.g., weekly, monthly, yearly, etc.) or intermittent therapy (e.g., interrupted treatment, intermittent treatment, treatment at relapse, or treatment upon achievement of a particular predetermined criteria [e.g., disease manifestation, etc.]).

As used herein, the term “hyaluronic acid” or “HA” refers to the polymer having the formula:

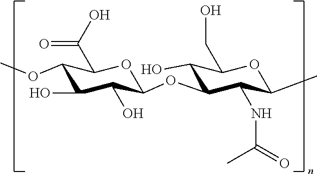

where n is the number of repeating units. All sources of hyaluronic acid are useful in this invention, including bacterial and avian sources. However, hyaluronic acid of bacterial origin is preferable. Hyaluronic acids useful in this invention have a molecular weight from 15,000 to 50,000 Daltons (“low molecular weight”). Preferably, the hyaluronic acid of the present invention has a molecular weight of 25,000 Daltons. In some embodiments, the hyaluronic acid of the present invention is administered to the subject in the form of a salt. In some embodiments, a salt of sodium, potassium, lithium, calcium, barium, strontium, magnesium, aluminium, or ammonium is used. In particular, the hyaluronic acid of the present invention is used in the form of a sodium salt. Commercial sources of hyaluronic acid typically include those from Sigma-Aldrich (e.g. CAS Number: 9067-32-7 or CAS Number: 9067-32-7).

By a “therapeutically effective amount” is meant a sufficient amount of the HA of the present invention for the treatment of the lung mucosal inflammation at a reasonable benefit/risk ratio applicable to any medical treatment. It will be understood that the total daily usage of the compound will be decided by the attending physician within the scope of sound medical judgment. The specific therapeutically effective dose level for any particular subject will depend upon a variety of factors including the age, body weight, general health, sex and diet of the subject; the time of administration, route of administration, and rate of excretion of the specific compound employed; the duration of the treatment; drugs used in combination or coincidental with the specific polypeptide employed; and like factors well known in the medical arts. For example, it is well known within the skill of the art to start doses of the compound at levels lower than those required to achieve the desired therapeutic effect and to gradually increase the dosage until the desired effect is achieved. However, the daily dosage of the products may be varied over a wide range from 0.01 to 1,000 mg per adult per day. Preferably, the compositions contain 0.01, 0.05, 0.1, 0.5, 1.0, 2.5, 5.0, 10.0, 15.0, 25.0, 50.0, 100, 250 and 500 mg of the active ingredient for the symptomatic adjustment of the dosage to the subject to be treated. A medicament typically contains from about 0.01 mg to about 500 mg of the active ingredient, preferably from 1 mg to about 100 mg of the active ingredient. An effective amount of the drug is ordinarily supplied at a dosage level from 0.0002 mg/kg to about 20 mg/kg of body weight per day, especially from about 0.001 mg/kg to 7 mg/kg of body weight per day.

Further to the reduction of lung mucosal inflammation, use of the hyaluronic acid of the present invention may combine additional effects. In particular, the hyaluronic acid of the present invention can regulate the differentiation of secretory cells and thus can participate in regulation the mucin secretion, and can modulate the secretion of chloride ions via CFTR and in particular via the F508del/F508del mutated CFTR.

In some embodiments, the hyaluronic acid is administered in combination with a corticosteroid. As used, the term “corticosteroid” has its general meaning in the art and refers to class of active ingredients having a hydrogenated cyclopentoperhydrophenanthrene ring system endowed with an anti-inflammatory activity. Corticosteroid drugs typically include cortisone, cortisol, hydrocortisone (11β,17-dihydroxy, 21-(phosphonooxy)-pregn-4-ene, 3,20-dione disodium), dihydroxycortisone, dexamethasone (21-(acetyloxy)-9-fluoro-1β,17-dihydroxy-16α-m-ethylpregna-1,4-diene-3,20-dione), and highly derivatized steroid drugs such as beconase (beclomethasone dipropionate, which is 9-chloro-11-β, 17,21, trihydroxy-16β-methylpregna-1,4 diene-3,20-dione 17,21-dipropionate). Other examples of corticosteroids include flunisolide, prednisone, prednisolone, methylprednisolone, triamcinolone, deflazacort and betamethasone. corticosteroids, for example, cortisone, hydrocortisone, methylprednisolone, prednisone, prednisolone, betamethesone, beclomethasone dipropionate, budesonide, dexamethasone sodium phosphate, flunisolide, fluticasone propionate, triamcinolone acetonide, betamethasone, fluocinolone, fluocinonide, betamethasone dipropionate, betamethasone valerate, desonide, desoximetasone, fluocinolone, triamcinolone, triamcinolone acetonide, clobetasol propionate, and dexamethasone.

Typically, the active ingredient of the present invention (i.e. the HA of the present invention) is combined with pharmaceutically acceptable excipients, and optionally sustained-release matrices, such as biodegradable polymers, to form pharmaceutical compositions. The term “Pharmaceutically” or “pharmaceutically acceptable” refers to molecular entities and compositions that do not produce an adverse, allergic or other untoward reaction when administered to a mammal, especially a human, as appropriate. A pharmaceutically acceptable carrier or excipient refers to a non-toxic solid, semi-solid or liquid filler, diluent, encapsulating material or formulation auxiliary of any type. The carrier can also be a solvent or dispersion medium containing, for example, water, ethanol, polyol (for example, glycerol, propylene glycol, and liquid polyethylene glycol, and the like), suitable mixtures thereof, and vegetables oils. The proper fluidity can be maintained, for example, by the use of a coating, such as lecithin, by the maintenance of the required particle size in the case of dispersion and by the use of surfactants. The prevention of the action of microorganisms can be brought about by various antibacterial and antifungal agents, for example, parabens, chlorobutanol, phenol, sorbic acid, thimerosal, and the like. In many cases, it will be preferable to include isotonic agents, for example, sugars or sodium chloride. Prolonged absorption of the injectable compositions can be brought about by the use in the compositions of agents delaying absorption, for example, aluminium monostearate and gelatin. In the pharmaceutical compositions of the present invention, the active ingredients of the invention can be administered in a unit administration form, as a mixture with conventional pharmaceutical supports. Suitable unit administration forms comprise oral-route forms such as tablets, gel capsules, powders, granules and oral suspensions or solutions, sublingual and buccal administration forms, aerosols, implants, subcutaneous, transdermal, topical, intraperitoneal, intramuscular, intravenous, subdermal, transdermal, intrathecal and intranasal administration forms and rectal administration forms. In some embodiments, the pharmaceutical composition of the invention is administered topically (i.e. in the respiratory tract of the subject). Therefore, the compositions can be formulated in the form of a spray, aerosol, solution, emulsion, or other form well-known to one of skill in the art. If the method of the invention comprises intranasal administration of a composition, the composition can be formulated in an aerosol form, spray, mist or in the form of drops. In particular, the active ingredients for use according to the present invention can be conveniently delivered in the form of an aerosol spray presentation from pressurized packs or a nebuliser, with the use of a suitable propellant (e.g., dichlorodifluoromethane, trichlorofluoromethane, dichlorotetrafluoroethane, carbon dioxide or other suitable gas). In the case of a pressurized aerosol the dosage unit may be determined by providing a valve to deliver a metered amount. Capsules and cartridges (composed of, e.g., gelatin) for use in an inhaler or insufflator may be formulated containing a powder mix of the compound and a suitable powder base such as lactose or starch.

The invention will be further illustrated by the following figures and examples. However, these examples and figures should not be interpreted in any way as limiting the scope of the present invention.

### FIGURES

FIG. 1: Measurement by ELISA assay of the pro-inflammatory chemokine IL-8 secreted by F508del/F508del CF human airway epithelial cells cultured at the air-liquid interface (ALI). Airway epithelial cells were seeded in bi-compartmental chambers and cultured in liquid-liquid condition until confluence was reached. At confluence, culture medium from the upper chamber was removed to create an air-liquid interface (ALI) that will favor epithelial cell differentiation. From the ALI creation, the different treatments (15-45 kDa sodium-hyaluronic acid (HA-Na 15-45 kDa) (ARD—Pomacle—France—as prepared according to WO2004050187), 15-30 kDa sodium-hyaluronic acid (HA-Na 15-30 kDa) (Sigma Aldrich) or 30-50 kDa sodium-hyaluronic acid (HA-Na 30-50 kDa) (Sigma Aldrich) were added at 1 mg/mL to the culture medium in the basal chamber. The culture medium was renewed three times a week. At ALI day 15, the culture medium was replaced by fresh medium without any treatment. After a 4 h-period, this culture medium was collected and the IL-8 content was determined by ELISA assay. n=5 different F508del/F508del CF patients. p<0.001 (***); p<0.01 (**); p<0.05 (*). ns: non-significant.

FIG. 2: Measurement by ELISA assay of the pro-inflammatory chemokine IL-8 secreted by F508del/F508del CF human airway epithelial cells cultured as monolayers. Airway epithelial cells were seeded in 48-wells plates and cultured until confluence was reached. Cells were then treated for 24 h with a pro-inflammatory chemokine cocktail (TNFα, IL1β and IFNγ; 10 ng/mL each) called Cytomix (Cy). Cells were thereafter treated with a combo containing Cytomix and the anti-inflammatory dexamethasone (Dexa; 10-6M), or 15-45 kDa sodium-hyaluronic acid (HA-Na 15-45 kDa) (ARD—Pomacle—France—as prepared according to WO2004050187), 15-30 kDa sodium-hyaluronic acid (HA-Na 15-30 kDa) (Sigma Aldrich), or 30-50 kDa sodium-hyaluronic acid (HA-Na 30-50 kDa) (Sigma Aldrich) at 1 mg/mL during the following 24 h. The culture medium was finally collected and the IL-8 content was determined by ELISA assay. n=7 different F508del/F508del CF patients. p<0.001 (***); p<0.01 (**); p<0.05 (*).ns: non-significant.

FIG. 3: Measurement by ELISA assay of the pro-inflammatory chemokine IL-8 secreted by F508del/F508del CF human airway epithelial cells cultured as monolayers. Airway epithelial cells were seeded in 48-wells plates and cultured until confluence was reached. Cells were then treated for 24 h with a combo containing a pro-inflammatory chemokine cocktail (TNFα, IL1β and IFNγ; 10 ng/mL each) called Cytomix (Cy) and the anti-inflammatory dexamethasone (Dexa; 10-6M), 15-45 kDa sodium-hyaluronic acid (HA-Na 15-45 kDa) (ARD—Pomacle—France—as prepared according to WO2004050187), 15-30 kDa sodium-hyaluronic acid (HA-Na 15-30 kDa) (Sigma Aldrich), or 30-50 kDa sodium-hyaluronic acid (HA-Na 30-50 kDa) (Sigma Aldrich) at 1 mg/mL. The culture medium was then collected and the IL-8 content was determined. n=7 F508del/F508del CF patient. p<0.001 (***); p<0.01 (**); p<0.05 (*). ns: non-significant.

FIG. 4: Measurement by ELISA assay of the pro-inflammatory chemokine IL-8 secreted by F508del/F508del CF human airway epithelial cells cultured at the air-liquid interface (ALI). Airway epithelial cells were seeded in bi-compartmental chambers and cultured in liquid-liquid condition until confluence was reached. At confluence, culture medium from the upper chamber was removed to create an air-liquid interface (ALI) that will favor epithelial cell differentiation. From the ALI creation, the different treatments (15-45 kDa sodium-hyaluronic acid (HA-Na 15-45 kDa) (ARD—Pomacle—France—as prepared according to WO2004050187), 15-45 kDa calcium-hyaluronic acid (HA-Ca 15-45 kDa; ARD), or 15-45 kDa potassium-hyaluronic acid (HA-K 15-45 kDa; ARD) were added at 1 mg/mL to the culture medium in the basal chamber. The culture medium was renewed three times a week. At ALI day 15, the culture medium was replaced by fresh medium without any treatment. After a 4 h-period, this culture medium was collected and the IL-8 content was determined by ELISA assay. n=4 different F508del/F508del CF patients. p<0.001 (***); p<0.01 (**); p<0.05 (*). ns: Non-significant.

FIG. 5: Measurement by ELISA assay of the pro-inflammatory chemokine IL-8 secreted by F508del/F508del CF human airway epithelial cells cultured as monolayers. Airway epithelial cells were seeded in 48-wells plates and cultured until confluence was reached. Cells were then treated for 24 h with a pro-inflammatory chemokine cocktail (TNFα, IL1β and IFNγ; 10 ng/ml each) called Cytomix (Cy). Cells were thereafter treated with a combo containing Cytomix and the anti-inflammatory dexamethasone (Dexa; 10-6M), or 15-45 kDa sodium-hyaluronic acid (HA-Na 15-45 kDa) (ARD—Pomacle—France—as prepared according to WO2004050187), 15-45 kDa calcium-hyaluronic acid (HA-Ca 15-45 kDa; ARD), or 15-45 kDa potassium-hyaluronic acid (HA-K 15-45 kDa; ARD) at 1 mg/mL during the following 24 h. The culture medium was finally collected and the IL-8 content was determined. n=7 different F508del/F508del CF patients. p<0.001 (***); p<0.01 (**); p<0.05 (*). ns: non-significant.

FIG. 6: Measurement by ELISA assay of the pro-inflammatory chemokine IL-8 secreted by F508del/F508del CF human airway epithelial cells cultured as monolayers. Airway epithelial cells were seeded in 48-wells plates and cultured until confluence was reached. Cells were then treated for 24 h with a combo containing a pro-inflammatory chemokine cocktail (TNFα, IL1β and IFNγ 10 ng/mL each) called Cytomix (Cy) and the anti-inflammatory dexamethasone (Dexa; 10-6M), or 15-45 kDa sodium-hyaluronic acid (HA-Na 15-45 kDa) (ARD—Pomacle—France—as prepared according to WO2004050187), 15-45 kDa calcium-hyaluronic acid (HA-Ca 15-45 kDa; ARD), or 15-45 kDa potassium-hyaluronic acid (HA-K 15-45 kDa; ARD) at 1 mg/mL. The culture medium was then collected and the IL-8 content was determined. n=7 different F508del/F508del CF patients. p<0.001 (***); p<0.01 (**); p<0.05 (*). ns: non-significant.

FIG. 7: Measurement by ELISA assay of the pro-inflammatory chemokine IL-8 secreted by F508del/F508del CF human airway epithelial cells cultured at the air-liquid interface (ALI). Airway epithelial cells were seeded in bi-compartmental chambers and cultured in liquid-liquid condition until confluence was reached. At confluence, culture medium from the upper chamber was removed to create an air-liquid interface (ALI) that will favor epithelial cell differentiation. From the ALI creation, 15-45 kDa sodium-hyaluronic acid (HA-Na 15-45 kDa) (ARD—Pomacle—France—as prepared according to WO2004050187) was added at 0.1 mg/mL, 0.5 mg/mL, 1 mg/mL and 2 mg/mL to the culture medium in the basal chamber. The culture medium was renewed three times a week. At ALI day 15, the culture medium was replaced by fresh medium without any treatment. After a 4 h-period, this culture medium was collected and the IL-8 content was determined by ELISA assay. n=3 different F508del/F508del CF patients. p<0.001 (***); p<0.01 (**); p<0.05 (*). ns: non-significant.

FIG. 8: Measurement by ELISA assay of the mucin MUC-5B secreted by F508del/F508del CF human airway epithelial cells cultured at the air-liquid interface (ALI). Airway epithelial cells were seeded in bi-compartmental chambers and cultured in liquid-liquid condition until confluence was reached. At confluence, culture medium from the upper chamber was removed to create an air-liquid interface (ALI) that will favor epithelial cell differentiation. From the ALI creation, the different treatments (15-45 kDa sodium-hyaluronic acid (HA-Na 15-45 kDa) (ARD—Pomacle—France—as prepared according to WO2004050187), 15-30 kDa sodium-hyaluronic acid (HA-Na 15-30 kDa) (Sigma Aldrich), or 30-50 kDa sodium-hyaluronic acid (HA-Na 30-50 kDa) (Sigma Aldrich)) were added at 1 mg/mL to the culture medium in the basal chamber. The culture medium was renewed three times a week. At ALI day 15 and ALI Day 25, the culture in the upper chamber was rinsed with PBS then, a small volume of fresh medium without any treatment was placed at the contact of the apical part of the epithelium. After a 4 h-period, this apical culture medium was collected and the MUC-5B content was determined by ELISA assay. n=3 different F508del/F508del CF patients. p<0.001 (***); p<0.01 (**); p<0.05 (*). ns: non-significant.

FIG. 9: Measurement by ELISA assay of the mucin MUC-5B secreted by F508del/F508del CF human airway epithelial cells cultured at the air-liquid interface (ALI). Airway epithelial cells were seeded in bi-compartmental chambers and cultured in liquid-liquid condition until confluence was reached. At confluence, culture medium from the upper chamber was removed to create an air-liquid interface (ALI) that will favor epithelial cell differentiation. From the ALI creation, the different treatments (15-45 kDa sodium-hyaluronic acid (HA-Na 15-45 kDa) (ARD—Pomacle—France—as prepared according to WO2004050187), 15-45 kDa calcium-hyaluronic acid (HA-Ca 15-45 kDa; ARD), or 15-45 kDa potassium-hyaluronic acid (HA-K 15-45 kDa; ARD) were added at 1 mg/mL to the culture medium in the basal chamber. The culture medium was renewed three times a week. At ALI day 15 and ALI Day 25, the culture in the upper chamber was rinsed with PBS then, a small volume of fresh medium without any treatment was placed at the contact of the apical part of the epithelium. After a 4 h-period, this apical culture medium was collected and the MUC-5B content was determined by ELISA assay. n=3 different F508del/F508del CF patients. p<0.001 (***); p<0.01 (**); p<0.05 (*). ns: non-significant.

FIG. 10: Measurement by ELISA assay of the mucin MUC-5B secreted by F508del/F508del CF human airway epithelial cells cultured at the air-liquid interface (ALI). Airway epithelial cells were seeded in bi-compartmental chambers and cultured in liquid-liquid condition until confluence was reached. At confluence, culture medium from the upper chamber was removed to create an air-liquid interface (ALI) that will favor epithelial cell differentiation. From the ALI creation, 15-45 kDa sodium-hyaluronic acid (HA-Na 15-45 kDa) (ARD—Pomacle—France—as prepared according to WO2004050187) was added at 2 mg/mL, 1 mg/mL or 0.5 mg/mL to the culture medium in the basal chamber. The culture medium was renewed three times a week. At ALI day 15 and ALI Day 25, the culture in the upper chamber was rinsed with PBS then, a small volume of fresh medium without any treatment was placed at the contact of the apical part of the epithelium. After a 4 h-period, this apical culture medium was collected and the MUC-5B content was determined by ELISA assay. n=3 different F508del/F508del CF patients. p<0.001 (***); p<0.01 (**); p<0.05 (*). ns: non-significant.

FIG. 11: Measurement by ELISA assay of the pro-inflammatory chemokine IL-8 secreted by non-CF human airway epithelial cells cultured at the air-liquid interface (ALI). Airway epithelial cells were seeded in bi-compartmental chambers and cultured in liquid-liquid condition until confluence was reached. At confluence, culture medium from the upper chamber was removed to create an air-liquid interface (ALI) that will favor epithelial cell differentiation. From the ALI creation, the different treatments (15-45 kDa sodium-hyaluronic acid (HA-Na 15-45 kDa) (ARD—Pomacle—France—as prepared according to WO2004050187), 15-30 kDa sodium-hyaluronic acid (HA-Na 15-30 kDa) (Sigma Aldrich), or 30-50 kDa sodium-hyaluronic acid (HA-Na 30-50 kDa) (Sigma Aldrich)) were added at 1 mg/mL to the culture medium in the basal chamber. The culture medium was renewed three times a week. At ALI day 15, the culture medium was replaced by fresh medium without any treatment. After a 4 h-period, this culture medium was collected and the IL-8 content was determined by ELISA assay. n=1 non-CF patient. p<0.001 (***); p<0.01 (**); p<0.05 (*). ns: non-significant.

FIG. 12: Measurement by ELISA assay of the pro-inflammatory chemokine IL-8 secreted non-CF human airway epithelial cells cultured as monolayers. Airway epithelial cells were seeded in 48-wells plates and cultured until confluence was reached. Cells were then treated for 24 h with a pro-inflammatory chemokine cocktail (TNFα, IL1β and IFNγ 10 ng/ml each) called Cytomix (Cy). Cells were thereafter treated with a combo containing Cytomix and the anti-inflammatory dexamethasone (Dexa; 10-6M), 15-45 kDa sodium-hyaluronic acid (HA-Na 15-45 kDa) (ARD—Pomacle—France—as prepared according to WO2004050187), 15-30 kDa sodium-hyaluronic acid (HA-Na 15-30 kDa) (Sigma Aldrich), or 30-50 kDa sodium-hyaluronic acid (HA-Na 30-50 kDa) (Sigma Aldrich) at 1 mg/mL during the following 24 h. The culture medium was finally collected and the IL-8 content was determined by ELISA assay. n=8 different non-CF patients. p<0.001 (***); p<0.01 (**); p<0.05 (*). ns: non-significant.

FIG. 13: Measurement by ELISA assay of the pro-inflammatory chemokine IL-8 secreted by non-CF human airway epithelial cells cultured as monolayers. Airway epithelial cells were seeded in 48-wells plates and cultured until confluence was reached. Cells were then treated for 24 h with a combo containing a pro-inflammatory chemokine cocktail (TNFα, IL1β and IFNγ 10 ng/ml each) called Cytomix (Cy) and the anti-inflammatory dexamethasone (Dexa; 10-6M), 15-45 kDa sodium-hyaluronic acid (HA-Na 15-45 kDa) (ARD—Pomacle—France—as prepared according to WO2004050187), 15-30 kDa sodium-hyaluronic acid (HA-Na 15-30 kDa) (Sigma Aldrich), or 30-50 kDa sodium-hyaluronic acid (HA-Na 30-50 kDa) (Sigma Aldrich) at 1 mg/mL. The culture medium was then collected and the IL-8 content was determined. n=8 different non-CF patients. p<0.001 (***); p<0.01 (**); p<0.05 (*). ns: non-significant.

FIG. 14: Measurement by ELISA assay of the pro-inflammatory chemokine IL-8 secreted by non-CF human airway epithelial cells cultured at the air-liquid interface (ALI). Airway epithelial cells were seeded in bi-compartmental chambers and cultured in liquid-liquid condition until confluence was reached. At confluence, culture medium from the upper chamber was removed to create an air-liquid interface (ALI) that will favor epithelial cell differentiation. From the ALI creation, the different treatments (15-45 kDa sodium-hyaluronic acid (HA-Na 15-45 kDa) (ARD—Pomacle—France—as prepared according to WO2004050187), 15-45 kDa calcium-hyaluronic acid (HA-Ca 15-45 kDa; ARD) or 15-45 kDa potassium-hyaluronic acid (HA-K 15-45 kDa; ARD) were added at 1 mg/mL to the culture medium in the basal chamber. The culture medium was renewed three times a week. At ALI day 15, the culture medium was replaced by fresh medium without any treatment. After a for 4 h-period, this culture medium was collected and the IL-8 content was determined by ELISA assay. n=1 non-CF patient. p<0.001 (***); p<0.01 (**); p<0.05 (*). ns: non-significant.

FIG. 15: Measurement by ELISA assay of the pro-inflammatory chemokine IL-8 secreted by non-CF human airway epithelial cells cultured as monolayers. Airway epithelial cells were seeded in 48-wells plates and cultured until confluence was reached. Cells were then treated for 24 h with a pro-inflammatory chemokine cocktail (TNFα, IL1β and IFNγ 10 ng/ml each) called Cytomix (Cy). Cells were thereafter treated with a combo containing Cytomix and the anti-inflammatory dexamethasone (Dexa; 10-6M), 15-45 kDa sodium-hyaluronic acid (HA-Na 15-45 kDa) (ARD—Pomacle—France—as prepared according to WO2004050187), 15-45 kDa calcium-hyaluronic acid (HA-Ca 15-45 kDa; ARD), or 15-45 kDa potassium hyaluronic acid (HA-K 15-45 kDa; ARD) at 1 mg/mL during the following 24 h. The culture medium was finally collected and the IL-8 content was determined. n=8 different non-CF patients. p<0.001 (***); p<0.01 (**); p<0.05 (*). ns: non-significant.

FIG. 16: Measurement by ELISA assay of the pro-inflammatory chemokine IL-8 secreted by non-CF human airway epithelial cells cultured as monolayers. Airway epithelial cells were seeded in 48-wells plates and cultured until confluence was reached. Cells were then treated for 24 h with a combo containing a pro-inflammatory chemokine cocktail (TNFα, IL1β and IFNγ 10 ng/ml each) called Cytomix (Cy) and the anti-inflammatory dexamethasone (Dexa; 10-6M), 15-45 kDa sodium-hyaluronic acid (HA-Na 15-45 kDa) (ARD—Pomacle—France—as prepared according to WO2004050187), 15-45 kDa calcium-hyaluronic acid (HA-Ca 15-45 kDa; ARD), or 15-45 kDa potassium hyaluronic acid (HA-K 15-45 kDa; ARD) at 1 mg/mL. The culture medium was then collected and the IL-8 content was determined. n=8 different non-CF patients. p<0.001 (***); p<0.01 (**); p<0.05 (*). ns: non-significant.

FIG. 17: Measurement by ELISA assay of the pro-inflammatory chemokine IL-8 secreted human COPD airway epithelial cells cultured as monolayers. Airway epithelial cells were seeded in 48-wells plates and cultured until confluence was reached. Cells were then treated for 24 h with a pro-inflammatory chemokine cocktail (TNFα, IL1β and IFNγ 10 ng/ml each) called Cytomix (Cy). Cells were thereafter treated with a combo containing Cytomix and the anti-inflammatory dexamethasone (Dexa; 10-6M), 15-45 kDa sodium-hyaluronic acid (HA-Na 15-45 kDa) (ARD—Pomacle—France—as prepared according to WO2004050187), 15-30 kDa sodium-hyaluronic acid (HA-Na 15-30 kDa) (Sigma Aldrich), or 30-50 kDa sodium-hyaluronic acid (HA-Na 30-50 kDa) (Sigma Aldrich) at 1 mg/mL during the following 24 h. The culture medium was finally collected and the IL-8 content was determined by ELISA assay. n=5 different COPD patients. p<0.001 (***); p<0.01 (**); p<0.05 (*). ns: non-significant.

FIG. 18: Measurement by ELISA assay of the pro-inflammatory chemokine IL-8 secreted by human COPD airway epithelial cells cultured as monolayers. Airway epithelial cells were seeded in 48-wells plates and cultured until confluence was reached. Cells were then treated for 24 h with a combo containing a pro-inflammatory chemokine cocktail (TNFα, IL1β and IFNγ 10 ng/mL each) called Cytomix (Cy) and the anti-inflammatory dexamethasone (Dexa; 10-6M), 15-45 kDa sodium-hyaluronic acid (HA-Na 15-45 kDa) (ARD—Pomacle—France—as prepared according to WO2004050187), 15-30 kDa sodium-hyaluronic acid (HA-Na 15-30 kDa) (Sigma Aldrich), or 30-50 kDa sodium-hyaluronic acid (HA-Na 30-50 kDa) (Sigma Aldrich) at 1 mg/mL. The culture medium was then collected and the IL-8 content was determined. n=5 COPD patient. p<0.001 (***); p<0.01 (**); p<0.05 (*). ns: non-significant.

FIG. 19: Measurement by ELISA assay of the pro-inflammatory chemokine IL-8 secreted by human COPD airway epithelial cells cultured as monolayers. Airway epithelial cells were seeded in 48-wells plates and cultured until confluence was reached. Cells were then treated for 24 h with a pro-inflammatory chemokine cocktail (TNFα, IL1β and IFNγ 10 ng/ml each) called Cytomix (Cy). Cells were thereafter treated with a combo containing Cytomix and the anti-inflammatory dexamethasone (Dexa; 10-6M), or 15-45 kDa sodium-hyaluronic acid (HA-Na 15-45 kDa) (ARD—Pomacle—France—as prepared according to WO2004050187), 15-45 kDa calcium-hyaluronic acid (HA-Ca 15-45 kDa; ARD), or 15-45 kDa potassium hyaluronic acid (HA-K 15-45 kDa; ARD) at 1 mg/mL during the following 24 h. The culture medium was finally collected and the IL-8 content was determined. n=5 different COPD patients. p<0.001 (***); p<0.01 (**); p<0.05 (*). ns: non-significant.

FIG. 20: Measurement by ELISA assay of the pro-inflammatory chemokine IL-8 secreted by human COPD airway epithelial cells cultured as monolayers. Airway epithelial cells were seeded in 48-wells plates and cultured until confluence was reached. Cells were then treated for 24 h with a combo containing a pro-inflammatory chemokine cocktail (TNFα, IL1β and IFNγ 10 ng/ml each) called Cytomix (Cy) and the anti-inflammatory dexamethasone (Dexa; 10-6M), 15-45 kDa sodium-hyaluronic acid (HA-Na 15-45 kDa) (ARD—Pomacle—France—as prepared according to WO2004050187), 15-45 kDa calcium-hyaluronic acid (HA-Ca 15-45 kDa; ARD), or 15-45 kDa potassium hyaluronic acid (HA-K 15-45 kDa; ARD) at 1 mg/mL. The culture medium was then collected and the IL-8 content was determined. n=5 different COPD patients. p<0.001 (***); p<0.01 (**); p<0.05 (*). ns: non-significant.

### EXAMPLE

Anti-Inflammatory Effects of Hyaluronic Acid of 15-50 kDa on Airway Epithelium in Patients Suffering from Cystic Fibrosis:

Sodium form of Hyaluronic Acid of 15-50 kDa exhibits an anti-inflammatory effect during the regeneration and differentiation of the human Cystic Fibrosis (CF) airway epithelium carrying the F508del/F508del class II mutation (FIG. 1). In particular, treatment with HA-Na 15-45 kDa leads to a significant decrease in IL-8 secretion (52%) by human CF airway epithelial cells, in comparison to control condition, as well as treatment with Ha-Na 15-30 kDa (57% decrease) and HA-Na 30-50 kDa (67.2% decrease).

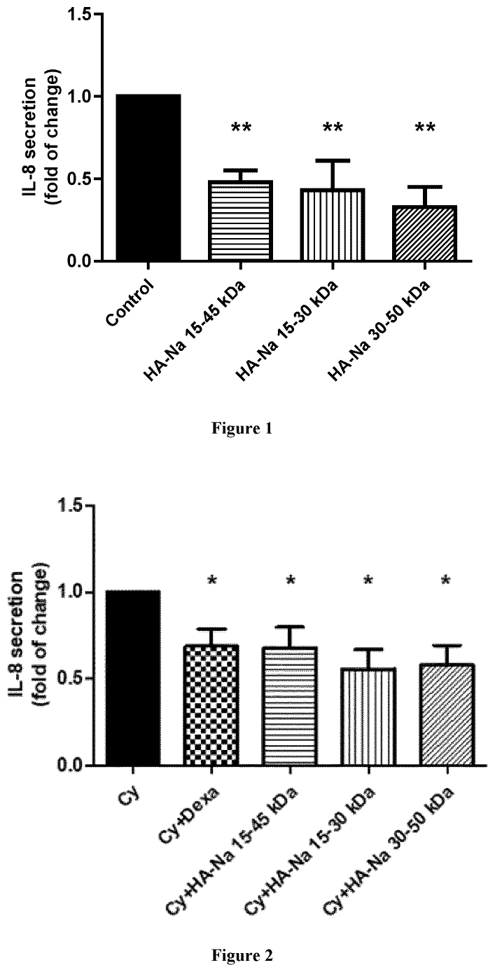

Sodium form of Hyaluronic Acid of 15-50 kDa exhibits a curative anti-inflammatory effect on the human Cystic Fibrosis (CF) airway epithelium carrying the F508del/F508del class II mutation (FIG. 2). In particular, treatment with HA-Na 15-45 kDa leads to a significant decrease in IL-8 secretion (32.1%) by human CF airway epithelial cells, as well as HA-Na 15-30 kDa (44.2% decrease), HA-Na 30-50 kDa (42.2% decrease) and the control anti-inflammatory Dexamethasone (30.9% decrease).

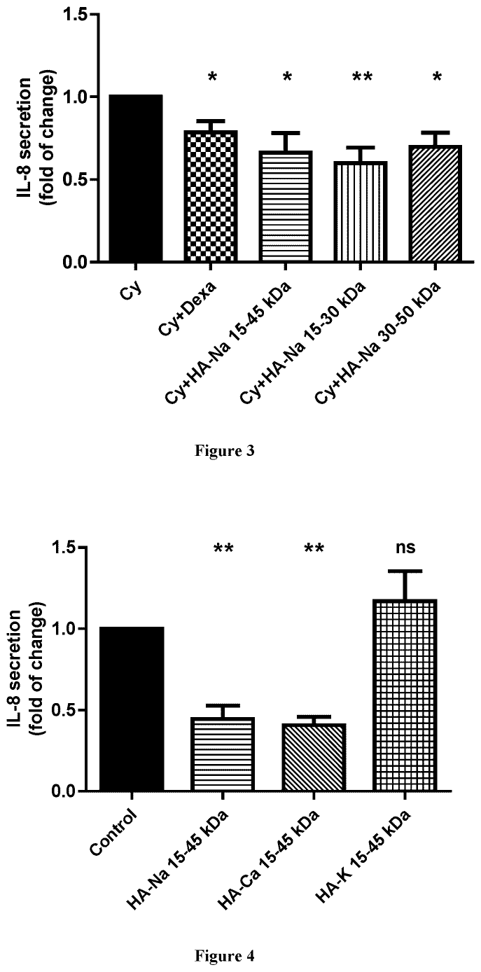

Sodium form of Hyaluronic Acid of 15-50 kDa exhibits an anti-inflammatory effect in co-treatment on the human Cystic Fibrosis (CF) airway epithelium carrying the F508del/F508del class II mutation (FIG. 3). In particular, treatment with HA-Na 15-45 kDa leads to a significant decrease in IL-8 secretion (33.9%) by human CF airway epithelial cells, as well as HA-Na 15-30 kDa (40.2% decrease), HA-Na 30-50 kDa (30.1% decrease) and the control anti-inflammatory Dexamethasone (21.4% decrease).

Hyaluronic Acid of 15-50 kDa exhibits an anti-inflammatory effect in its sodium and calcium forms during the regeneration and differentiation of the human Cystic Fibrosis (CF) airway epithelium carrying the F508del/F508del class II mutation (FIG. 4). In particular, treatment with HA-Na 15-45 kDa leads to a significant decrease in IL-8 secretion (55.5%) by human CF airway epithelial cells, in comparison to control condition, as well as treatment with Ha-Ca 15-45 kDa (59.25% decrease).

Hyaluronic Acid of 15-50 kDa exhibits a curative anti-inflammatory effect in its sodium and potassium forms on the human Cystic Fibrosis (CF) airway epithelium carrying the F508del/F508del class II mutation (FIG. 5). In particular, treatment with 15-45 kDa sodium-hyaluronic acid (HA-Na) leads to a significant decrease in IL-8 secretion (32.1%) by human CF airway epithelial cells, as well as Dexamethasone (30.9% decrease) and 15-45 kDa potassium-hyaluronic acids (47.5% decrease). 15-45 kDa calcium-hyaluronic acids leads to a 33.6% non-significant decrease in IL-8 secretion.

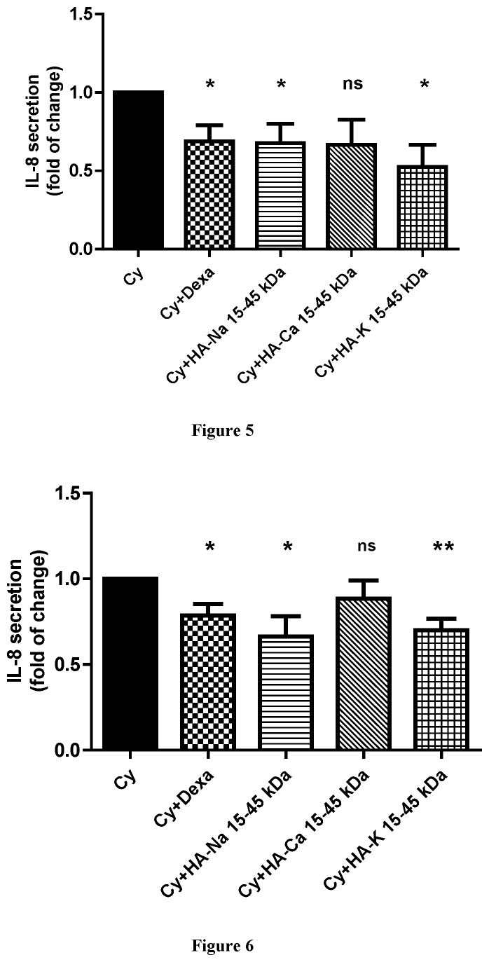

Hyaluronic Acid of 15-50 kDa exhibits an anti-inflammatory effect in co-treatment in its sodium and potassium forms on the human Cystic Fibrosis (CF) airway epithelium carrying the F508del/F508del class II mutation (FIG. 6). In particular, treatment with 15-45 kDa sodium-hyaluronic acid (HA-Na 15-45 kDa) leads to a significant decrease in IL-8 secretion (33.9%) by human CF airway epithelial cells, as well as Dexamethasone (21.4% decrease) and 15-45 kDa potassium-hyaluronic acids (30% decrease). 15-45 kDa calcium-hyaluronic acids leads to a 11.6% non-significant decrease in IL-8 secretion.

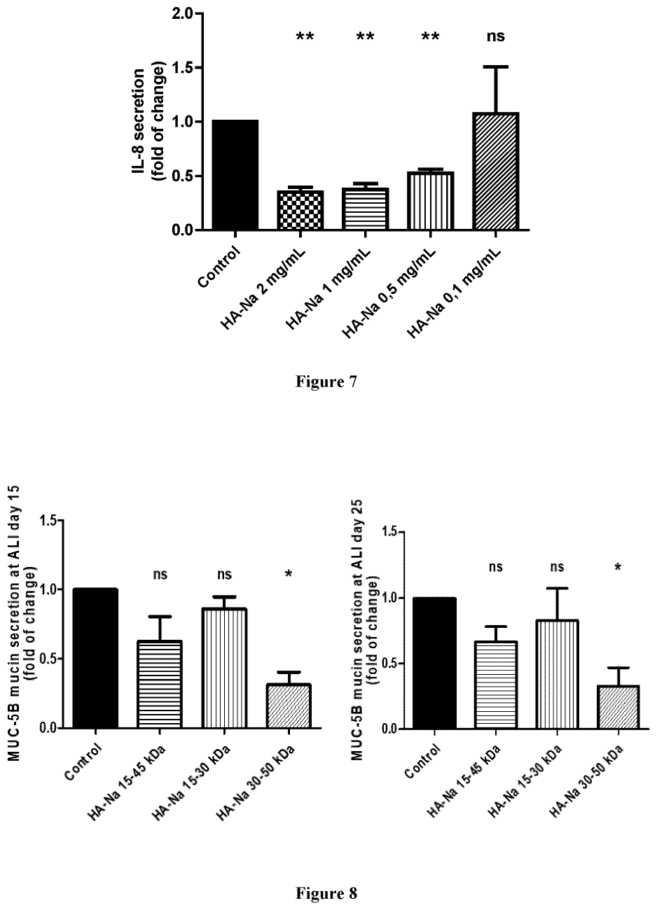

Sodium form of Hyaluronic Acid of 15-50 kDa exhibits a dose-response anti-inflammatory effect during the regeneration and differentiation of the human Cystic Fibrosis (CF) airway epithelium carrying the F508del/F508del class II mutation (FIG. 7). In particular, treatment with 15-45 kDa sodium-hyaluronic acid (HA-Na) leads to a significant decrease in IL-8 secretion by human CF airway epithelial cells at 2 mg/mL (65% decrease), 1 mg/mL (62% decrease), 0.5 mg/mL (47.3%) and does not have anymore effect at 0.1 mg/mL.

Sodium form of Hyaluronic Acid of 15-50 kDa leads to a decrease in the MUC-5B mucin secretion during the regeneration of the human Cystic Fibrosis (CF) airway epithelium carrying the F508del/F508del class II mutation (FIG. 8). In particular, treatment with HA-Na 30-50 kDa leads to a significant decrease in MUC-5B secretion (ALI day 15:68.7%; ALI Day 25:67.1%) by human CF airway epithelial cells, in comparison to control condition whereas treatment with HA-Na 15-45 kDa and HA-Na 15-30 kDa leads to a non-significant decrease in MUC-5B secretion (ALI day 15: 37.3% and 14.1%, respectively; ALI Day 25: 33.2% and 17.3%, respectively).

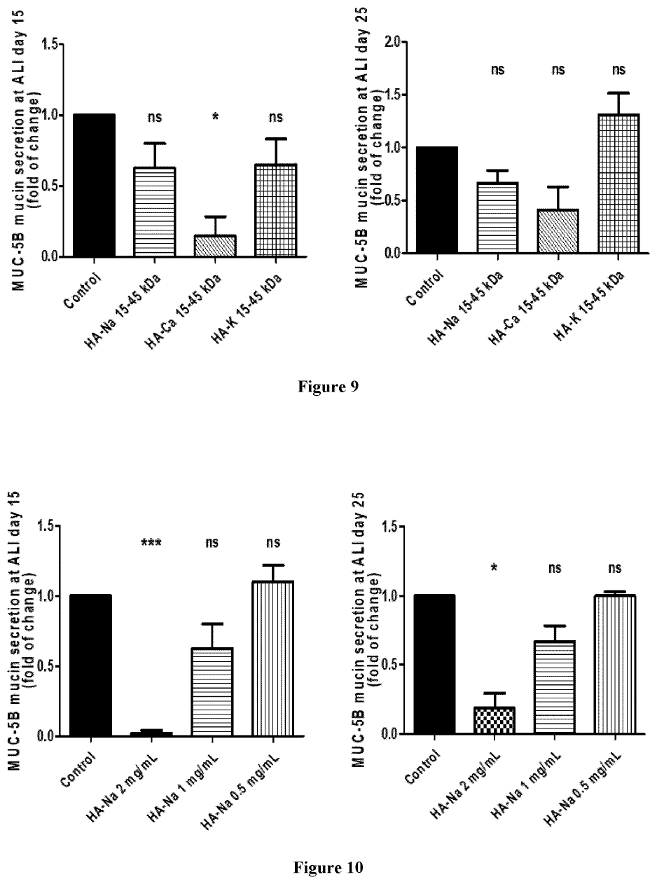

Hyaluronic Acid of 15-50 kDa leads to a decrease in the MUC-5B mucin secretion in its sodium, calcium and potassium forms during the regeneration of the human Cystic Fibrosis (CF) airway epithelium carrying the F508del/F508del class II mutation (FIG. 9). In particular, treatment with HA-Ca 15-45 kDa leads to a significant decrease at ALI day 15 (85.5% decrease) and to a non-significant decrease at ALI day 25 (58.8%) in MUC-5B secretion by human CF airway epithelial cells, in comparison to control condition. Treatment with HA-Na 15-45 kDa and HA-K 15-45 kDa leads to a non-significant decrease in MUC-5B secretion at ALI day 15 (37.3% and 34.7% decrease, respectively) in comparison to control condition. Treatment with HA-Na 15-45 kDa leads to a non-significant decrease in MUC-5B secretion at ALI day 25 (33.2% decrease) by human CF airway epithelial cells, in comparison to control condition.

Sodium form of Hyaluronic Acid of 15-50 kDa leads to a dose-dependent decrease in the MUC-5B mucin secretion during the regeneration of the human Cystic Fibrosis (CF) airway epithelium carrying the F508del/F508del class II mutation (FIG. 10). In particular, treatment with HA-Na 15-45 kDa leads to a significant decrease in MUC-5B secretion by human CF airway epithelial cells at ALI Day 15 and ALI day 25 at 2 mg/mL (97.9% and 81.2% decrease, respectively), to a non-significant decrease in MUC-5B secretion at 1 mg/mL (ALI day 15: 37.3% decrease; ALI day 25: 33.2% decrease) but does not have any effect at 0.5 mg/mL, in comparison to control condition.

### Anti-Inflammatory Effects of Hyaluronic Acid of 15-50 kDa on Airway Epithelium in Non-CF Patients:

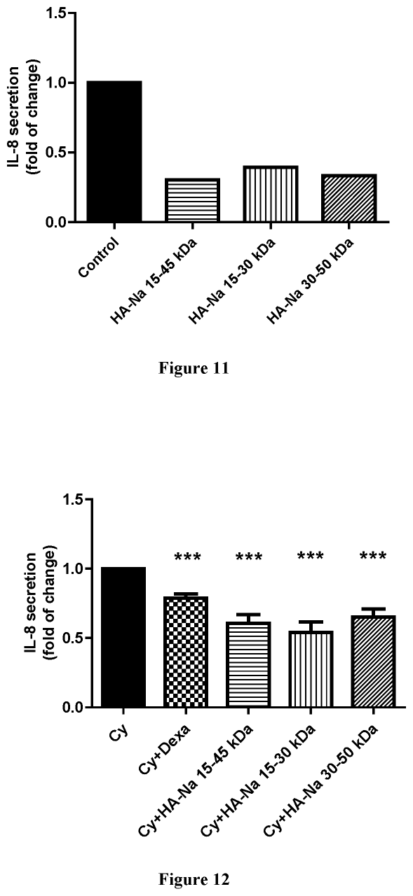

Sodium form of Hyaluronic Acid of 15-50 kDa exhibits an anti-inflammatory effect during the regeneration and differentiation of the human airway epithelium of non-CF patients (FIG. 11). In particular, treatment with HA-Na 15-45 kDa leads to a decrease in IL-8 secretion (69.7%) by human non-CF airway epithelial cells, in comparison to control condition, as well as treatment with HA-Na 15-30 (60.6% decrease) and HA-Na 30-50 kDa (66.6% decrease).

Sodium form of Hyaluronic Acid of 15-50 kDa exhibits a curative anti-inflammatory effect on the human airway epithelium of non-CF patients suffering from lung inflammation (FIG. 12). In particular, treatment with HA-Na 15-45 kDa leads to a significant decrease in IL-8 secretion (39.6%) by human non-CF airway epithelial cells, as well as HA-Na 15-30 kDa (46.1% decrease), HA-Na 30-50 kDa (34.8% decrease) and the control anti-inflammatory Dexamethasone (21.1% decrease).

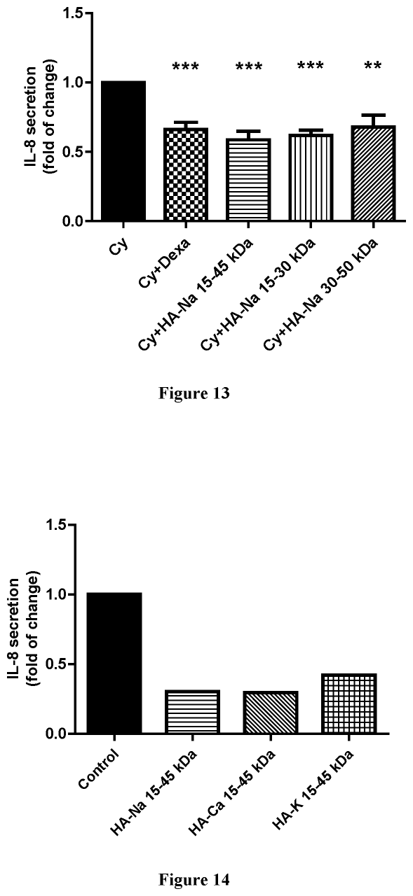

Sodium form of Hyaluronic Acid of 15-50 kDa exhibits an anti-inflammatory effect in co-treatment on the human airway epithelium of non-CF patients suffering from lung inflammation (FIG. 13). In particular, treatment with HA-Na 15-45 kDa leads to a significant decrease in IL-8 secretion (41.6%) by human non-CF airway epithelial cells, as well as HA-Na 15-30 kDa (38.1% decrease), HA-Na 30-50 kDa (32.1% decrease) and the control anti-inflammatory Dexamethasone (33.9% decrease).

Hyaluronic Acid of 15-50 kDa exhibits an anti-inflammatory effect in its sodium, calcium and potassium forms during the regeneration and differentiation of the human airway epithelium of non-CF patients (FIG. 14). In particular, treatment with HA-Na 15-45 kDa leads to a decrease in IL-8 secretion (69.7%) by human non-CF airway epithelial cells, in comparison to control condition, as well as treatment with Ha-Ca 15-45 kDa (70.5% decrease) and HA-K 15-45 kDa (57.9% decrease).

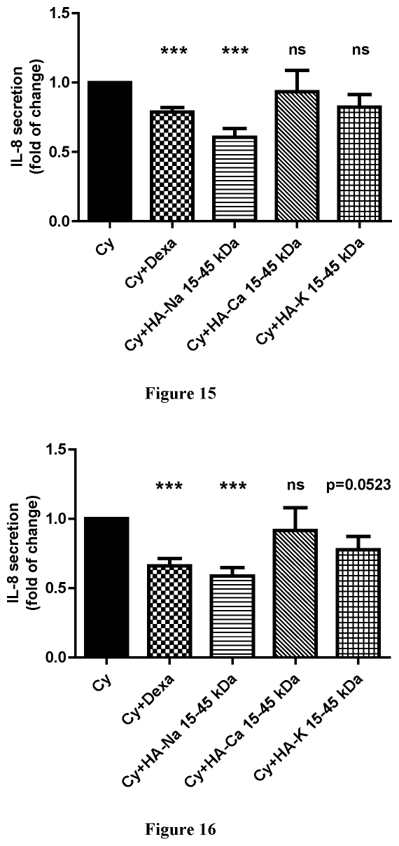

Hyaluronic Acid of 15-50 kDa exhibits a curative anti-inflammatory effect in its sodium form on the human airway epithelium of non-CF patients suffering from lung inflammation (FIG. 15). In particular, treatment with 15-45 kDa sodium-hyaluronic acid (HA-Na) leads to a significant decrease in IL-8 secretion (39.6%) by human non-CF airway epithelial cells, as well as Dexamethasone (21.1% decrease). 15-45 kDa calcium-hyaluronic acids and 15-45 kDa potassium-hyaluronic acids lead to non-significant decreases (6.5% and 17.8%, respectively) in IL-8 secretion.

Hyaluronic Acid of 15-50 kDa exhibits an anti-inflammatory effect in co-treatment in its sodium form on the human airway epithelium of non-CF patients suffering from lung inflammation (FIG. 16). In particular, treatment with 15-45 kDa sodium-hyaluronic acid (HA-Na 15-45 kDa) leads to a significant decrease in IL-8 secretion (41.6%) by human non-CF airway epithelial cells, as well as Dexamethasone (33.9% decrease). 15-45 kDa calcium-hyaluronic acids and 15-45 kDa potassium-hyaluronic acids lead to non-significant decreases (8.5% and 22.2%, respectively) in IL-8 secretion.

Anti-Inflammatory Effects of Hyaluronic Acid of 15-50 kDa on Airway Epithelium in Patients Suffering from COPD:

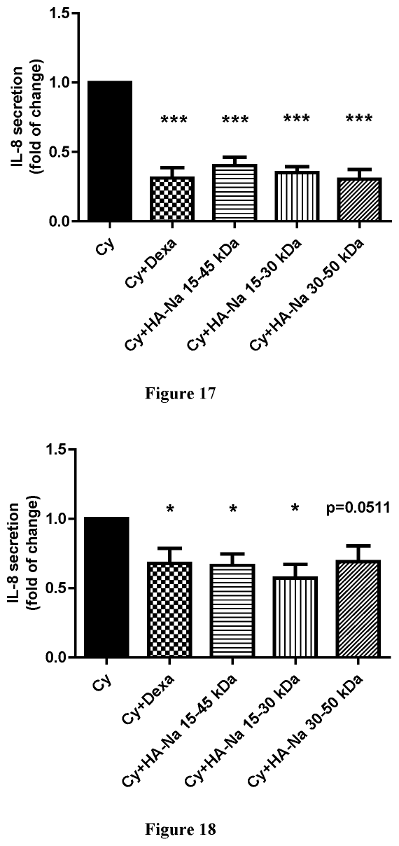

Sodium form of Hyaluronic Acid of 15-50 kDa exhibits a curative anti-inflammatory effect on the human airway epithelium of COPD patients (FIG. 17). In particular, treatment with HA-Na 15-45 kDa leads to a significant decrease in IL-8 secretion (60.2%) by human COPD airway epithelial cells, as well as HA-Na 15-30 kDa (65% decrease), HA-Na 30-50 kDa (69.5% decrease) and the control anti-inflammatory Dexamethasone (68.8% decrease).

Sodium form of Hyaluronic Acid of 15-50 kDa exhibits an anti-inflammatory effect in co-treatment on the human airway epithelium of COPD patients (FIG. 18). In particular, treatment with HA-Na 15-45 kDa leads to a significant decrease in IL-8 secretion (33.4%) by human COPD airway epithelial cells, as well as HA-Na 15-30 kDa (43.1% decrease), and the control anti-inflammatory Dexamethasone (32.1% decrease). HA-Na 30-50 kDa leads to a non-significant (p=0.0511) decrease in IL-8 secretion (30.8% decrease).

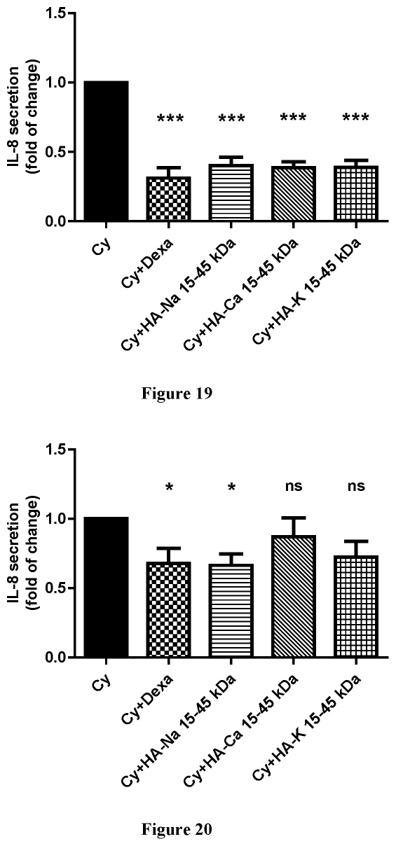

Hyaluronic Acid of 15-50 kDa exhibits a curative anti-inflammatory effect in its sodium, calcium and potassium forms on the human airway epithelium of COPD patients (FIG. 19). In particular, treatment with 15-45 kDa sodium-hyaluronic acid (HA-Na) leads to a significant decrease in IL-8 secretion (60.2%) by human COPD airway epithelial cells, as well as Dexamethasone (68.8% decrease) and 15-45 kDa calcium- or potassium-hyaluronic acids (HA-Ca 15-45 kDa: 61.5% decrease; HA-K 15-45 kDa: 61.1% decrease).

Hyaluronic Acid of 15-50 kDa exhibits an anti-inflammatory effect in co-treatment in its sodium form on the human airway epithelium of COPD patients (FIG. 20). In particular, treatment with 15-45 kDa sodium-hyaluronic acid (HA-Na 15-45 kDa) leads to a significant decrease in IL-8 secretion (33.4%) by human COPD airway epithelial cells, as well as Dexamethasone (32.1% decrease). 15-45 kDa calcium- or potassium-hyaluronic acids lead to a non-significant decrease (12.9% decrease and 27.5% decrease, respectively) in IL-8 secretion.

### REFERENCES

Throughout this application, various references describe the state of the art to which this invention pertains. The disclosures of these references are hereby incorporated by reference into the present disclosure.

1. A method of treating lung mucosal inflammation in a subject suffering from cystic fibrosis or chronic obstructive pulmonary disease comprising administering to the subject a therapeutically effective amount of hyaluronic acid having a low molecular weight, from 15,000 to 50,000 Daltons.

2. The method of claim 1 wherein the subject suffering from cystic fibrosis harbors at least one mutation in the CFTR gene.

3. The method of claim 1 wherein the lung mucosal inflammation results from a lung infection.

4. The method of claim 3 wherein the subject suffers from COVID-19.

5. The method of claim 1 wherein the hyaluronic acid is administered to the subject in the form of a salt.

6. The method of claim 5 wherein the salt is a salt of sodium, potassium, lithium, calcium, barium, strontium, magnesium, aluminium, or ammonium.

7. The method of claim 5 wherein the hyaluronic acid is in the form of a sodium salt.

8. The method of claim 1 wherein the hyaluronic regulates the differentiation of secretory cells, participates in regulation the mucin secretion, and/or modulates the secretion of chloride ions via CFTR.

9. The method of claim 1 wherein the hyaluronic acid is administered in combination with a corticosteroid.

10. The method of claim 9 wherein the corticosteroid is selected from the group consisting of flunisolide, prednisone, prednisolone, methylprednisolone, triamcinolone, deflazacort, betamethasone, cortisone, hydrocortisone, methylprednisolone, prednisone, prednisolone, betamethesone, beclomethasone dipropionate, budesonide, dexamethasone sodium phosphate, flunisolide, fluticasone propionate, triamcinolone acetonide, betamethasone, fluocinolone, fluocinonide, betamethasone dipropionate, betamethasone valerate, desonide, desoximetasone, fluocinolone, triamcinolone, triamcinolone acetonide, clobetasol propionate, and dexamethasone.

11. The method of claim 2, wherein the at least one mutation in the CFTR gene is F508del-CFTR, R117H CFTR, or G551D CFTR.

12. The method of claim 3 wherein the lung infection is a viral infection.

13. The method of claim 8 wherein the CFTR is F508del/F508del mutated CFTR.
